SEQUENCE LISTING (55 sequences)

SEQ ID NO: 1 - protein, 8 aa, Homo sapiens
VVLGAHNV

SEQ ID NO: 2 - protein, 8 aa, Homo sapiens
VLGAHNVR

SEQ ID NO: 3 - protein, 8 aa, Homo sapiens
LGAHNVRT

SEQ ID NO: 4 - protein, 8 aa, Homo sapiens
GAHNVRTQ

SEQ ID NO: 5 - protein, 8 aa, Homo sapiens
PHSRPYMA

SEQ ID NO: 6 - protein, 8 aa, Homo sapiens
QPHSRPYM

SEQ ID NO: 7 - protein, 8 aa, Homo sapiens
HSRPYMAS

SEQ ID NO: 8 - protein, 8 aa, Homo sapiens
FLNNYDAE

SEQ ID NO: 9 - protein, 8 aa, Homo sapiens
CFGDSGGP

SEQ ID NO: 10 - protein, 8 aa, Homo sapiens
MAHRPPSP

SEQ ID NO: 11 - protein, 8 aa, Homo sapiens
AHRPPSPA

SEQ ID NO: 12 - protein, 8 aa, Homo sapiens
HRPPSPAL

SEQ ID NO: 13 - protein, 8 aa, Homo sapiens
AQPHSRPY

SEQ ID NO: 14 - protein, 8 aa, Homo sapiens
PGSHFCGG

SEQ ID NO: 15 - protein, 8 aa, Homo sapiens
PVPHGTQC

SEQ ID NO: 16 - protein, 7 aa, Homo sapiens
FCRPHNI

SEQ ID NO: 17 - protein, 7 aa, Homo sapiens
PRRKAGI

SEQ ID NO: 18 - protein, 7 aa, Homo sapiens
ATVQLPQ

SEQ ID NO: 19 - protein, 7 aa, Homo sapiens
QLPQQDQ

SEQ ID NO: 20 - protein, 7 aa, Homo sapiens
PVPHGTQ

SEQ ID NO: 21 - protein, 7 aa, Homo sapiens
QPHSRPY

SEQ ID NO: 22 - protein, 7 aa, Homo sapiens
RVGAHDP

SEQ ID NO: 23 - protein, 7 aa, Homo sapiens
IDSFVIW

SEQ ID NO: 24 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 234 aa, synthetic construct
LLQGARSDATHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 241 aa, synthetic construct
AHGHGHGLLQGARSDATHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 4 aa, synthetic construct
LLQG

SEQ ID NO: 28 - protein, 256 aa, Homo sapiens
MAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 29 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 30 - protein, 119 aa, Homo sapiens
MKLVRFLMKLSHETVTIELKNGTQVHGTITGVDVSMNTHLKAVKMTLKNREPVQLETLSIRGNNIRYFILPDSLPLDTLLVDVEPKVKSKKREAVAGRGRGRGRGRGRGRGRGRGGPRR

SEQ ID NO: 31 - protein, 14 aa, Homo sapiens
AHCLRDIPQRLVNV

SEQ ID NO: 32 - protein, 16 aa, Homo sapiens
HGTQCLAMGWGRVGAH

SEQ ID NO: 33 - protein, 14 aa, Homo sapiens
NICTFVPRRKAGIC

SEQ ID NO: 34 - protein, 6 aa, Homo sapiens
SRPYMA

SEQ ID NO: 35 - protein, 15 aa, Homo sapiens
ATRLFPDFFTRVALY

SEQ ID NO: 36 - protein, 37 aa, Homo sapiens
VEPKVKSKKREAVAGRGRGRGRGRGRGRGRGRGGPRR

SEQ ID NO: 37 - protein, 25 aa, Homo sapiens
VAGRGRGRGRGRGRGRGRGRGGPRR

SEQ ID NO: 38 - protein, 15 aa, Homo sapiens
MPELTSMHTLLLREH

SEQ ID NO: 39 - protein, 15 aa, Homo sapiens
PILRGLMATPAKLNR

SEQ ID NO: 40 - protein, 11 aa, Homo sapiens
DHGLPGYNAWR

SEQ ID NO: 41 - protein, 12 aa, Homo sapiens
FPTDQLTPDQER

SEQ ID NO: 42 - protein, 10 aa, Homo sapiens
IANVFTNAFR

SEQ ID NO: 43 - protein, 12 aa, Homo sapiens
IGLDLPALNMQR

SEQ ID NO: 44 - protein, 10 aa, Homo sapiens
IPCFLAGDTR

SEQ ID NO: 45 - protein, 7 aa, Homo sapiens
LFEQVMR

SEQ ID NO: 46 - protein, 11 aa, Homo sapiens
NNIFMSNTYPR

SEQ ID NO: 47 - protein, 13 aa, Homo sapiens
RKIVGAMVQIITY

SEQ ID NO: 48 - protein, 10 aa, Homo sapiens
RSPTLGASNR

SEQ ID NO: 49 - protein, 7 aa, Homo sapiens
VFFASWR

SEQ ID NO: 50 - protein, 12 aa, Homo sapiens
VVLEGGIDPILR

SEQ ID NO: 51 - protein, 15 aa, Homo sapiens
RKIVGAMVQIITYRD

SEQ ID NO: 52 - protein, 14 aa, Homo sapiens
GHEAQPHSRPYMAS

SEQ ID NO: 53 - protein, 13 aa, Homo sapiens
NVVLGAHNVRTQE

SEQ ID NO: 54 - protein, 17 aa, Homo sapiens
ATVQLPQQDQPVPHGTQ

SEQ ID NO: 55 - protein, 17 aa, Homo sapiens
HGTQCLAMGWGRVGAHD

(19) United States
(12) Patent Application Publication
(10) Pub. No.: US 2026/0364195 A1
(43) Pub. Date: Nov. 19, 2026

(54) Hybrid Molecule Comprising an Antibody FC Portion and at Least One Peptide Binding to a Self-Reactive Lymphocyte Involved in Autoimmune Vasculitis, and Uses Thereof

(71) Applicants: UNIVERSITE TOULOUSE III – PAUL SABATIER, Toulouse (FR); INSTITUT NATIONAL DE LA SANTÉ ET DE LA RECHERCHE MÉDICALE, Paris (FR); CENTRE HOSPITALIER UNIVERSITAIRE DE TOULOUSE, Toulouse (FR); CENTRE HOSPITALIER UNIVERSITAIRE DE MONTPELLIER, Montpellier (FR); UNIVERSITE DE MONTPELLIER, Montpellier (FR); ARTHRITIS RECHERCHE & DEVELOPPEMENT, Neuilly sur Seine (FR)

(72) Inventors: Christian Jorgensen, Saint-Clement de Riviere (FR); Eve Combes, Villeveyrac (FR); Jennifer Lambour, Montpellier (FR)

(73) Assignees: UNIVERSITE TOULOUSE III – PAUL SABATIER, Toulouse (FR); INSTITUT NATIONAL DE LA SANTÉ ET DE LA RECHERCHE MÉDICALE, Paris (FR); CENTRE HOSPITALIER UNIVERSITAIRE DE TOULOUSE, Toulouse (FR); CENTRE HOSPITALIER UNIVERSITAIRE DE MONTPELLIER, Montpellier (FR); UNIVERSITE DE MONTPELLIER, Montpellier (FR); ARTHRITIS RECHERCHE & DEVELOPPEMENT, Neuilly sur Seine (FR)

(21) Appl. No.: 19/111,936

(22) PCT Filed: Sep. 15, 2023
(86) PCT No.: PCT/FR2023/051404

§ 371 Date: Mar. 14, 2025

(30) Foreign Application Priority Data
Sep. 15, 2022 (FR) 2209319

(51) Int. Cl. A61K 47/68; A61P 37/06; C12N 9/08; C12N 9/64

(52) U.S. Cl. CPC A61K 47/6815; A61K 47/6889; A61P 37/06; C12N 9/0065; C12N 9/6424; C12Y 111/02002; C12Y 304/21076; C07K 2319/30

(57) ABSTRACT

The invention relates to a hybrid molecule comprising at least one antibody Fc fragment covalently bonded to at least one peptide bonding to an autoreactive lymphocyte, involved in autoimmune vasculitis, the uses of such a hybrid molecule, as well as the production method thereof.

The present invention relates to a hybrid molecule comprising at least one antibody Fc fragment covalently bonded to at least one peptide bonding to an autoreactive lymphocyte, involved in autoimmune vasculitis, the uses of such a hybrid molecule, as well as the production method thereof. Said autoreactive lymphocyte receptor is a membrane receptor on the surface of a B-lymphocyte (BCR) or on the surface of a T-lymphocyte (TCR).

### BACKGROUND OF THE INVENTION

Autoimmune vasculitis is a rare disease characterized by inflammation of the walls of small blood vessels in the body, in particular linked to the production of autoantibodies by the body and/or pro-inflammatory cytokines secreted by T-lymphocytes.

These autoantibodies are directed in particular against certain white blood cells: polymorphonuclear neutrophils and can therefore sometimes be called ANCA (anti-neutrophil cytoplasmic antibodies).

These autoantibodies are often used to diagnose autoimmune vasculitis and its role in the pathogenesis of the disease has been studied (e.g., Bruner B F, Vista E S, Wynn D M, Harley J B, James J A. Anti-neutrophil cytoplasmic antibodies target sequential functional proteinase 3 epitopes in the sera of patients with Wegener's granulomatosis. Clin Exp Immunol. 2010 November; 162(2):262-70. doi: 10.1111/j.1365-2249.2010.04251.x. PMID: 21077276; PMCID: PMC2996593; or Van Der Geld Y M, Simpelaar A, Van Der Zee R, Tervaert J W, Stegeman C A, Limburg P C, Kallenberg C G. Anti-neutrophil cytoplasmic antibodies to proteinase 3 in Wegener's granulomatosis: epitope analysis using synthetic peptides. Kidney Int. 2001 January; 59(1):147-59. doi: 10.1046/j.1523-1755.2001.00475.x. PMID: 11135067). These autoantibodies are therefore a key therapeutic target.

The antigenic targets of these autoantibodies involved in autoimmune vasculitis have been profiled and can be found in the IEDB database, Immune Epitope DataBase and analysis resource, http://www.iedb.org/home_v3.php. These autoantibodies are in particular directed specifically against proteinase 3 (PR3), or specifically against myeloperoxidase (MPO) or against the small nuclear ribonucleoprotein SmD1 (“small nuclear ribonucleoprotein”).

Autoimmune vasculitis (or “ANCA-positive vasculitis” or ANCA-associated vasculitis) mainly groups together three diseases: granulomatosis with polyangiitis (sometimes called Wegener's disease), eosinophilic granulomatosis with polyangiitis (sometimes called Churg-Strauss syndrome) and micropolyangiitis. Granulomatosis with polyangiitis is characterized by inflammatory necrosis of small and medium-sized vessels (capillaries, venules and arterioles) which leads to tissue ischemia. Eosinophilic granulomatosis with polyangiitis is defined as a systemic vasculitis of small vessels that is also characterized by asthma, blood and tissue eosinophilia; and micropolyangiitis is defined as a systemic inflammatory necrotizing vasculitis that primarily affects small vessels of multiple organs (small arteries, arterioles, capillaries, venules).

To this day, there is no specific treatment for autoimmune vasculitis. Treatments usually involve administering corticosteroids or even immunosuppressive drugs, which can cause serious side effects. Anti-CD20 antibodies may also be used, but these are not specific for the B-cells behind autoreactive antibodies and have no effect on autoreactive T-cells.

An object of the present invention is thus to provide a more targeted treatment of autoimmune vasculitis. By targeting B-cells behind autoreactive antibodies (autoantibodies) associated with autoimmune vasculitis, and/or autoreactive T-lymphocytes behind pro-inflammatory cytokines, the present invention thus aims to provide a more specific treatment.

The present invention is based on the Inventors' research showing that it is possible to target the autoreactive lymphocyte receptors that recognize self peptides involved in the development of autoimmune vasculitis. More particularly, the present invention is based on the Inventors' research showing that it is possible to target the B-lymphocyte clones expressing the lymphocyte receptors involved in autoimmune vasculitis and/or autoreactive T-lymphocytes (by bonding a self peptide involved in the development of autoimmune vasculitis to the TCR), and to eliminate them using a hybrid molecule comprising (i) at least one peptide bonding to an autoreactive lymphocyte involved in autoimmune vasculitis and (ii) a human immunoglobulin Fc fragment. These hybrid molecules will specifically target B-lymphocyte clones expressing autoantibodies responsible for autoimmune vasculitis and/or autoreactive T-lymphocytes (using said peptide, which is recognized by said B- and/or T-receptors expressed by said B- and/or T-lymphocytes) which will then be eliminated, after binding the Fc fragment to the Fc receptors, by macrophages (via phagocytosis) and/or NK-cells (via antibody-dependent cell-mediated cytotoxicity-ADCC), and/or by complement cascade activation.

By targeting B-lymphocyte clones expressing autoantibodies responsible for autoimmune vasculitis and cells that differentiate into plasma cells that themselves secrete said autoantibodies and/or autoreactive T-lymphocytes sources of pro-inflammatory cytokines, the hybrid molecules of the invention thus aim to eliminate these “pathogenic” autoantibodies from the patients' bodies.

### DISCLOSURE OF THE INVENTION

### Hybrid Molecule According to the Invention

In a first aspect, the invention relates to a hybrid molecule comprising at least one antibody Fc fragment covalently bonded to at least one peptide bonding to an autoreactive lymphocyte involved in autoimmune vasculitis, at least one spacer being optionally present between said Fc fragment and said peptide. The outline of such a structure is shown in FIG. 1.

According to the invention, a “hybrid molecule” means a molecule having at least two components of different nature, namely the antibody Fc fragment and said peptide.

According to the invention, an “Fc fragment” of an antibody means the constant region of an immunoglobulin excluding the first immunoglobulin constant region domain (i.e. CH1-CL). Thus, the Fc fragment refers to a homodimer, each monomer comprising the last two constant domains of IgA, IgD, IgG (i.e. CH2 and CH3), or the last three constant domains of IgE and IgM (i.e., CH2, CH3 and CH4).

According to the invention, the expression “covalently bonded” means a covalent bond, that is to say a chemical bond in which two atoms share two electrons. Said covalent bond can be polar or non-polar.

According to the invention, a “spacer” is a bonding agent which makes it possible to covalently bond an Fc fragment of an antibody to said peptide bonding to an autoreactive lymphocyte involved in autoimmune vasculitis, while distancing said Fc fragment from said peptide (thereby reducing possible steric hindrance). It can be any molecule, and in particular a peptide or a polypeptide. Preferably, the spacer does not modify the physicochemical properties of the hybrid molecule.

The presence of at least one spacer is advantageous: it facilitates the independent accessibility of the two partners of the hybrid molecule (the Fc fragment is more easily accessible to bond with the Fc receptors, just as said peptide is more easily accessible to bond with the autoreactive lymphocytes), and/or stabilizes the hybrid molecule, and/or increases the solubility of the hybrid molecule.

According to an embodiment, the hybrid molecule according to the invention may comprise one or more spacers. Preferably, the hybrid molecule comprises one or two spacers. According to an embodiment, when at least one spacer is present in said hybrid molecule of the invention, said Fc fragment is covalently bonded to a spacer, said spacer itself being covalently bonded to said peptide. According to another embodiment, when at least two spacers are present in said hybrid molecule of the invention, said Fc fragment is covalently bonded to a first spacer, said first spacer itself being covalently bonded to a second spacer and the second spacer itself is covalently bonded to said peptide. The bond between the Fc fragment and the peptide can therefore be direct, or indirect in the presence of spacers.

According to an embodiment, the hybrid molecule according to the invention can comprise at least one peptide bonding to an autoreactive lymphocyte involved in autoimmune vasculitis. This means that the Fc fragment can be bonded to one or two peptides. Indeed, the Fc fragment comprises two monomers, and the Fc fragment can thus be covalently bonded to a peptide on only one of the two monomers, or the Fc fragment can be covalently bonded to a peptide on each monomer. Preferably, when two peptides are bonded with the Fc fragment, the two peptides are identical.

According to an embodiment, said spacer is a polymer containing one or more repeating units containing the ether group. According to a particular embodiment, said spacer is polyethylene glycol of formula PEG-n, wherein n denotes an integer between 1 and 100, preferably between 1 and 10, and in particular 1, 2, 3, 4 or 8. According to the invention, said polyethylene glycol may be functionalized, for example with an amine group (PEG-n-amine such as PEG-NH₂). According to the invention “an integer between 1 and 100” encompasses all integer values between 1 and 100, i.e.; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100.

The expression “autoreactive lymphocyte receptor involved in autoimmune vasculitis” means a receptor on the surface of a T-lymphocyte and/or a B-lymphocyte that recognizes a self peptide involving the activation of cells leading to autoimmune vasculitis by inappropriate production of autoantibodies or pro-inflammatory cytokines. More particularly, said lymphocyte receptor is a B-lymphocyte receptor (BCR) and/or a T-lymphocyte receptor (TCR). The bond of the peptide to the BCR and/or TCR is thus involved in the development of autoimmune vasculitis. For example, and according to an embodiment, the involved self peptide may comprise all or part of proteinase 3 and/or an “autoantibody responsible for autoimmune vasculitis” means an “anti-neutrophil cytoplasm autoantibody (ANCA)”. Typically, binding these ACPNs to their target leads to activation of neutrophils with increased adhesion to the vascular endothelium, production of oxygen free radicals and release of enzymes behind tissue damage.

The term “autoreactive lymphocyte involved in autoimmune vasculitis” means a lymphocyte expressing on its surface a receptor that recognizes a self peptide involving the activation of cells leading to autoimmune vasculitis by inappropriate production of autoantibodies or pro-inflammatory cytokines. Preferably, it is a T-lymphocyte and/or a B-lymphocyte.

According to a preferred embodiment, an autoantibody according to the invention means an autoantibody directed against proteinase 3 (anti-PR3), an autoantibody directed against myeloperoxidase (anti-MPO) and/or an autoantibody directed against the small nuclear ribonucleoprotein SmD1 (anti-SmD1). Preferably, the autoantibodies are anti-neutrophil cytoplasm autoantibodies such as anti-PR3 (c-ANCA for cytoplasmic-ANCA) or anti-MPO (p-ANCA for perinuclear-ANCA). According to an even more preferred embodiment, an autoantibody according to the invention means an autoantibody directed against proteinase 3 (anti-PR3), and the autoantibodies are anti-neutrophil cytoplasm autoantibodies such as anti-PR3 (c-ANCA for cytoplasmic-ANCA).

The term “autoimmune vasculitis” means more specifically necrotizing inflammation of small vessels, with little or no immune complex deposition, and the frequent presence of circulating antibodies directed against proteinase 3 (PR3) and/or against myeloperoxidase (anti-MPO) and/or against the small nuclear ribonucleoprotein SmD1 (anti-SmD1).

According to an embodiment, the expression “peptide bonding to an autoreactive lymphocyte involved in autoimmune vasculitis” means a peptide comprising all or part of the polypeptide sequence of proteinase 3, all or part of the polypeptide sequence of myeloperoxidase and/or all or part of the polypeptide sequence of the small nuclear ribonucleoprotein SmD1, preferably a peptide comprising all or part of the polypeptide sequence of proteinase 3.

In an embodiment, the expression “peptide bonding to an autoreactive lymphocyte involved in autoimmune vasculitis” means a peptide comprising all or part of the polypeptide sequence of proteinase 3. Preferably, said proteinase 3 polypeptide sequence is represented by SEQ ID NO: 28.

In an embodiment, the expression “peptide bonding to an autoreactive lymphocyte involved in autoimmune vasculitis” means a peptide comprising all or part of the polypeptide sequence of myeloperoxidase. Preferably, said myeloperoxidase polypeptide sequence is represented by SEQ ID NO: 29.

In an embodiment, the term “peptide bonding to an autoreactive lymphocyte involved in autoimmune vasculitis” means a peptide comprising all or part of the polypeptide sequence of the small nuclear ribonucleoprotein SmD1. Preferably, said polypeptide sequence of said SmD1 is represented by SEQ ID NO: 30.

In an embodiment, a “peptide bonding to an autoreactive lymphocyte involved in autoimmune vasculitis” means a peptide recognized by an autoantibody directed against proteinase 3, against myeloperoxidase and/or against the small nuclear ribonucleoprotein SmD1. Such peptides can be obtained from fragments of proteinase, myeloperoxidase and/or the small nuclear ribonucleoprotein SmD1, whether these fragments are natural, recombinant or synthetic. Such peptides can also be directly synthesized. The amino acids constituting the peptide may be of the L or D series, preferably of the L series. A peptide according to the invention bonds to an autoantibody directed against proteinase 3, against myeloperoxidase and/or against the small nuclear ribonucleoprotein SmD1, and the bond between said peptide and the autoantibody can for example be verified using an ELISA test. See also, for example, Csernok, E., Moosig, F. Current and emerging techniques for ANCA detection in vasculitis. Nat Rev Rheumatol 10, 494-501 (2014). https://doi.org/10.1038/nrrheum.2014.78.

According to an embodiment, in said hybrid molecule according to the invention, said peptide comprises all or part of the polypeptide sequence of proteinase 3, all or part of the polypeptide sequence of myeloperoxidase and/or all or part of the polypeptide sequence of the small nuclear ribonucleoprotein SmD1, and said proteinase 3, myeloperoxidase and/or SmD1 is of mammalian origin and is preferably of human origin. According to an embodiment, in said hybrid molecule according to the invention, said peptide comprises all or part of the polypeptide sequence of proteinase 3, all or part of the polypeptide sequence of myeloperoxidase or all or part of the polypeptide sequence of the small nuclear ribonucleoprotein SmD1, and said proteinase 3, myeloperoxidase or SmD1 is of mammalian origin and is preferably of human origin.

According to an embodiment, in said hybrid molecule according to the invention, the peptide has a size of at least 2 consecutive amino acids, 3 consecutive amino acids, 4 consecutive amino acids, preferably at least 5 consecutive amino acids. According to an embodiment, said peptide has a size of between 5 and 70 amino acids. According to the invention, “between 5 and 70” means all values: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70. According to an embodiment, in said hybrid molecule according to the invention, the peptide has a size of between 5 and 15 consecutive amino acids, preferably between 5 and 10.

According to an embodiment, in said hybrid molecule according to the invention, the peptide is linear.

According to an embodiment, in said hybrid molecule according to the invention, the peptide can be modified so as to improve its reactivity towards autoantibodies. For example, peptides can be cyclized, peptides can be of the retro type (the L series amino acids are chained in a sequence that is the reverse of that of the peptide to replicate), or of the retro-inverso type (the amino acids are of the D type instead of the natural L series and are chained in a sequence that is the reverse of that of the peptide to replicate). According to an even more particular embodiment, in said hybrid molecule according to the invention, the terminal carboxyl function (COOH) of said peptide is replaced by a carboxamide function (CONH₂).

According to another embodiment, in said hybrid molecule according to the invention, the peptide can be modified so as to facilitate its synthesis and/or improve its stability, for example by alkylation. According to an even more particular embodiment, in said hybrid molecule according to the invention, the terminal amine function (NH₂) of said peptide is acetylated.

According to an embodiment, in said hybrid molecule according to the invention, the amine and carboxyl functions of the peptide can be in the form of the salt corresponding to the acid or to the base.

According to an even more particular embodiment, in said hybrid molecule according to the invention, said peptide is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50 and SEQ ID NO: 51.

According to an even more particular embodiment, in said hybrid molecule according to the invention, said peptide is selected from the group consisting of: SEQ ID NO: 52, SEQ ID NO: 53, SEQ ID NO: 54 and SEQ ID NO: 55.

According to an even more particular embodiment, in said hybrid molecule according to the invention, said peptide is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 and SEQ ID NO: 55.

According to an even more particular embodiment, in said hybrid molecule according to the invention, said peptide is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 31, SEQ ID NO: 32, SEQ ID NO: 33, SEQ ID NO: 34 and SEQ ID NO: 35 (PR3).

According to an even more particular embodiment, in said hybrid molecule according to the invention, said peptide is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 31, SEQ ID NO: 32, SEQ ID NO: 33, SEQ ID NO: 34, SEQ ID NO: 35, SEQ ID NO: 52, SEQ ID NO: 53, SEQ ID NO: 54 and SEQ ID NO: 55 (PR3).

According to an even more particular embodiment, in said hybrid molecule according to the invention, said peptide is selected from the group consisting of: SEQ ID NO: 36 and SEQ ID NO: 37 (SmD1).

According to an even more particular embodiment, in said hybrid molecule according to the invention, said peptide is selected from the group consisting of: SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50 and SEQ ID NO: 51 (MPO).

According to an embodiment, in said hybrid molecule according to the invention, said Fc fragment is a human Fc fragment, in particular of IgG, more particularly of IgG1. IgG1 can correspond to any allotypic variant, for example G1m3 or nG1m17. For example, the Fc fragment of IgG1 is represented by SEQ ID NO: 24, SEQ ID NO: 25 (Fc+Qtag) or SEQ ID NO: 26 (Fc+Qtag bis).

According to an embodiment, in said hybrid molecule according to the invention, said Fc fragment is a wild-type or mutated fragment. The mutation(s) may aim to increase the plasma half-life, decrease it, or modify the effector functions of the Fc fragment. According to an even more particular embodiment, said mutated Fc fragment comprises at least the following mutations:
L234A and L235A (LALA), or
L234A, L235A and P329G (LALAPG), or
G236A, S239D and 1332E (GASDIE), or
G236A, S239D, A330L and I332E (GASDALIE), or
S239D, H268F, S324T and 1332E (SDHFSTIE or SDH),
the numbering being indicated in the sequence of a human IgG1 according to the EU index. Such mutations are in particular described in the Bruhns and Jonsson article, Immunol Rev. 2015 November; 268(1):25-51. Preferably, when the hybrid molecule is used in therapy, said mutated Fc fragment comprises at least the GASDIE, GASDALIE, or SDH mutations.

According to an embodiment, in said hybrid molecule according to the invention, said Fc fragment has a fucosylation level of between 0% and 100% of the glycosylated forms. According to the invention “between 0% and 100%” encompasses all integer values between 0 and 100, i.e.: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%. Low fucosylation of the Fc fragment elicits a strong ADCC response. This is why, according to a particular embodiment, said Fc fragment has a fucosylation level of between 0% and 60% of the glycosylated forms, in particular 50%, 40%, 30%, 20%, 10% or 0%. According to the invention, the fucosylation level is defined as the average proportion of fucose carried by the Fc fragment, relative to the maximum quantity of fucose that an Fc fragment can carry.

The Fc fragment and the peptide each have N- and C-terminal ends. The Fc fragment can therefore be bonded via its N- or C-terminal end to the N- or C-terminal end of the peptide. According to a preferred embodiment, in said hybrid molecule according to the invention, said covalent bond is located between the C-terminal end of said Fc fragment and the N-terminal end of said peptide, or between the N-terminal end of said Fc fragment and the N-terminal end of said peptide. According to an embodiment, when a spacer is present, the spacer can be bonded to the Fc fragment via its N- or C-terminal end. According to another embodiment, when two spacers are present, the first spacer can be bonded to the Fc fragment via its N- or C-terminal end and the second spacer can be bonded to the peptide via its N- or C-terminal end, in particular its N-terminal end. Alternatively, said covalent bond between said Fc fragment and said peptide (optionally in the presence of one or more spacers) can be created on all or part of the Fc fragment. According to the invention, “all or part of the Fc fragment” means that different amino acids constituting the Fc fragment may be involved in a covalent bond with said peptide.

According to a preferred embodiment, when at least one spacer is present in said hybrid molecule of the invention, this makes it possible to bond the Fc fragment to an azide or to an alkyne which will itself be involved in the covalent bond with said peptide. According to an embodiment, when at least one spacer is present in said hybrid molecule of the invention, the at least one spacer can also make it possible to bond the peptide to an alkyne or to an azide which will itself be involved in the covalent bond with the Fc fragment. According to a preferred embodiment, the hybrid molecule according to the invention comprises at least two spacers: a first spacer which makes it possible to bond the Fc fragment to an azide or to an alkyne, and a second spacer which makes it possible to bond the peptide to an azide (when the Fc fragment is bonded to an alkyne) or to an alkyne (when the Fc fragment is bonded to an azide).

According to an embodiment according to the invention, in said hybrid molecule according to the invention, said Fc fragment:
is coupled to at least one azide or an alkyne, such as a cyclooctyne, and in particular DBCO, or
is bonded to at least one spacer which is itself coupled to an azide or an alkyne, such as a cyclooctyne, and in particular DBCO, and said peptide is:
either coupled to an azide or to an alkyne, such as a cyclooctyne, and in particular DBCO,
either bonded to a spacer which is itself coupled to an azide or to an alkyne, such as a cyclooctyne, and in particular DBCO,
the covalent bond between said Fc fragment and said peptide, optionally in the presence of one or more spacers, being created between the azide and the alkyne.

According to an embodiment according to the invention, in said hybrid molecule according to the invention,
the Fc fragment is coupled to at least one azide and said peptide is coupled to an alkyne, such as a cyclooctyne, and in particular DBCO, or
the Fc fragment is coupled to at least one alkyne, such as a cyclooctyne, and in particular DBCO, and said peptide is coupled to an azide, or
the covalent bond between said Fc fragment and said peptide being created between the azide and the alkyne.

According to an embodiment according to the invention, in said hybrid molecule according to the invention:
the Fc fragment is coupled to at least one azide and said peptide is bonded to a spacer, itself coupled to an alkyne, such as a cyclooctyne, and in particular DBCO or
the Fc fragment is coupled to at least one alkyne, such as a cyclooctyne, and in particular DBCO, and said peptide is bonded to a spacer, itself coupled to an azide or
the Fc fragment is bonded to at least one spacer, itself coupled to an azide, and said peptide is coupled to an alkyne, such as a cyclooctyne, and in particular DBCO or
the Fc fragment is bonded to at least one spacer, itself coupled to an alkyne, such as a cyclooctyne, and in particular DBCO, and said peptide is coupled to an azide or
the Fc fragment is bonded to at least one spacer, itself coupled to an azide, and said peptide is bonded to a spacer, itself coupled to an alkyne, such as a cyclooctyne, and in particular DBCO or
the Fc fragment is bonded to at least one spacer, itself coupled to an alkyne, such as a cyclooctyne, and in particular DBCO, and said peptide is bonded to a spacer, itself coupled to an azide,
the covalent bond between said Fc fragment and said peptide, in the presence of one or more spacers, being created between the azide and the alkyne.

According to the invention, the creation of the covalent bond between the azide and the alkyne corresponds to a step named a “click chemistry” step, wherein the N₃ part of the azide reacts with an alkyne. Azide means salts of hydrozoic acid HN₃, or organic azides in which one of the nitrogen atoms is covalently bonded to a carbon atom of an organic compound (e.g., methyl azide CH₃N₃). Preferably the azide is represented by the formula N₃. Alkynes are molecules with the general formula CₙH₂ₙ₋₂, which are characterized by the presence of at least one triple bond. Preferably the alkyne is a cyclooctyne, even more preferably dibenzocyclooctyne (DBCO).

The Fc fragment, said peptide and optionally said spacer(s), are coupled to the alkyne or to the azide by any conventionally used molecular coupling technique (such as conjugation). Any technique can also be used to covalently bond the Fc fragment to the spacer and/or the peptide to the spacer.

More specifically, a conjugation technique means an enzymatic conjugation or a chemical conjugation. An enzymatic conjugation means, for example, a conjugation using a transglutaminase which catalyzes the formation of covalent bonds between free amine groups and glutamine or lysine residues or using a transpeptidase such as sortase. For further information on enzymatic conjugation, see, e.g., patent application US20160361434 or US20170313787, or the publication by Ohtsuka et al., Bioscience, Biotechnology, and Biochemistry Volume 64, 2000-Issue 12, Comparison of Substrate Specificities of Transglutaminases Using Synthetic Peptides as Acyl donors. The substrate of the transglutaminase is for example a peptide comprising a glutamyl residue (a Qtag), as represented by SEQ ID NO: 27 (LLQG). A chemical conjugation means, for example, a covalent bond between an isolated cysteine or one part of a disulfide bridge after reduction of the latter and, for example, a maleimide. An example of such a conjugation is shown in FIG. 4. In this example, the Fc fragment comprises a Qtag peptide and said Fc fragment is bonded to a spacer (itself coupled to an azide), thanks to the action of transglutaminase which will create a covalent bond between the glutamyl residue of the Qtag and the N_{H2} group carried by the PEGn spacer.

According to the invention, the term “coupled” or “molecular coupling” means the establishment of a covalent bond, thus the Fc fragment and/or the peptide and/or the spacer is covalently bonded to an alkyne or an azide. The term “bonded” also applies to a covalent bond. Thus, by way of example, the expression “the Fc fragment is coupled to an azide and said peptide is bonded to a spacer, itself coupled to an alkyne, such as a cyclooctyne, and in particular DBCO” can also be read as “the Fc fragment is covalently bonded to an azide and said peptide is covalently bonded to a spacer, said spacer itself being covalently bonded to an alkyne, such as a cyclooctyne, and in particular DBCO”.

### Use of Hybrid Molecules According to the Invention

In a second aspect, the present invention also relates to a hybrid molecule as defined above, for its use as a drug.

More particularly, according to the invention, said hybrid molecules are intended to target and lyse in the body of patients, by ADCC and/or phagocytosis and/or complement activation, all the cells expressing autoreactive lymphocyte receptors within the framework of autoimmune vasculitis: namely the B-cells (lymphocytes) expressing on their surface the BCRs recognizing at least one of the peptides of the invention (autoantibodies responsible for autoimmune vasculitis, bonding to at least one peptide according to the invention) and T-lymphocytes expressing on their surface the TCRs recognizing at least one of the peptides according to the invention. Indeed, the hybrid molecule according to the invention bonds to these cells thanks to the peptide: this is the epitope target of said autoreactive lymphocyte receptor. The hybrid molecule according to the invention also bonds to cells making it possible to destroy B and/or T-lymphocytes expressing on their surface the autoreactive lymphocyte receptors involved in autoimmune vasculitis thanks to its Fc fragment, a natural ligand for Fc receptors (for example Fc-gamma receptor (FcγR) if the Fc fragment comes from an IgG), present in particular on the surface of macrophages but also of NK-cells (“Natural Killers”).

According to a particular embodiment, the invention relates to a hybrid molecule, as defined above, for its use in the treatment of autoimmune vasculitis, in particular granulomatosis with polyangiitis, eosinophilic granulomatosis with polyangiitis and micropolyangiitis. According to a preferred embodiment, the invention relates to a hybrid molecule, as defined above, for its use in the treatment of granulomatosis with polyangiitis.

According to a particular embodiment, the invention relates to a hybrid molecule as defined above and comprising a peptide selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50 and SEQ ID NO: 51, for its use in the treatment of autoimmune vasculitis, preferably granulomatosis with polyangiitis.

According to a particular embodiment, the invention relates to a hybrid molecule as defined above and comprising a peptide selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 and SEQ ID NO: 55, for its use in the treatment of autoimmune vasculitis.

According to a particular embodiment, the invention relates to a hybrid molecule as defined above and comprising a peptide selected from the group consisting of: SEQ ID NO: 2, SEQ ID NO: 7, SEQ ID NO: 31, SEQ ID NO: 52, SEQ ID NO: 53, SEQ ID NO: 54 and SEQ ID NO: 55, for its use in the treatment of autoimmune vasculitis. According to a particular embodiment, the invention relates to a hybrid molecule as defined above and comprising a peptide selected from the group consisting of: SEQ ID NO: 2, SEQ ID NO: 7, SEQ ID NO: 31, SEQ ID NO: 52, SEQ ID NO: 53, SEQ ID NO: 54 and SEQ ID NO: 55, for use in the treatment of granulomatosis with polyangiitis (sometimes called Wegener's disease), eosinophilic granulomatosis with polyangiitis (sometimes called Churg-Strauss syndrome), or micropolyangiitis.

According to an embodiment, the invention also relates to a pharmaceutical composition comprising a hybrid molecule according to any one of the preceding claims, in combination with a pharmaceutically acceptable carrier.

According to the invention, “a pharmaceutically acceptable carrier” means any formulation making the composition suitable for its administration to a patient, in any galenic form.

The present invention also relates to a method for treating autoimmune vasculitis, in particular granulomatosis with polyangiitis, comprising administering a therapeutically effective amount of a hybrid molecule according to the invention.

The invention also relates to the use of a hybrid molecule according to the invention for preparing a drug intended for treating autoimmune vasculitis, in particular granulomatosis with polyangiitis.

According to another embodiment, the hybrid molecule according to the invention may be coupled to at least one radioisotope or to at least one fluorochrome, such as A488 or A647. Such molecules can advantageously be used as molecular tracer tools.

According to another embodiment, the invention thus relates to the in vitro or ex vivo use of a hybrid molecule comprising at least one antibody Fc fragment covalently bonded to at least one peptide bonding to an autoreactive lymphocyte involved in autoimmune vasculitis, at least one spacer being optionally present between said Fc fragment and said peptide, as a molecular tool. Such structures can be used in particular to analyze binding of hybrid molecules to the autoantibodies and to the Fc receptors of macrophages and NK cells, as well as to analyze the reactivity of macrophages and NK cells to the binding of the hybrids followed by the bridging thereof by the autoantibodies. . . . Radioisotopes and/or fluorochromes are preferably coupled to the Fc fragment, even more particularly at the level of the Qtag (where available) or at the level of the lysines.

### Method for Producing the Hybrid Molecules According to the Invention

In another aspect, the invention also relates to a method for obtaining a hybrid molecule as defined above.

According to an embodiment, the invention thus relates to a method for producing a hybrid molecule as defined above, comprising the following steps:
(ii) obtaining an azide coupled to an Fc fragment or obtaining an alkyne coupled to an Fc fragment, wherein
(ii) obtaining an alkyne coupled to a peptide or obtaining an azide coupled to a peptide, wherein
(iii) carrying out the covalent bond between the azide and the alkyne,
step (i) can be carried out before or after step (ii), or at the same time.

According to an embodiment, the invention thus relates to a method for producing a hybrid molecule as defined above, comprising the following steps:
(i) coupling at least one azide and one Fc fragment, optionally in the presence of at least one spacer, or coupling at least one alkyne and at least one Fc fragment, optionally in the presence of a spacer,
(ii) coupling an alkyne and a peptide, optionally in the presence of a spacer, or coupling an azide and a peptide, optionally in the presence of a spacer,
(iii) carrying out the covalent bond between the azide and the alkyne,
step (i) can be carried out before or after step (ii), or at the same time.

According to another embodiment, the invention thus relates to a method for producing a hybrid molecule as defined above, comprising the following steps:
(i) coupling at least one azide to each monomer of the Fc fragment, optionally in the presence of at least one spacer, or coupling at least one alkyne to each monomer of the Fc fragment, optionally in the presence of a spacer,
(ii) coupling an alkyne and a peptide, optionally in the presence of a spacer, or coupling an azide and a peptide, optionally in the presence of a spacer,
(iii) carrying out the covalent bond(s) between the azide and the alkyne,
step (i) can be carried out before or after step (ii), or at the same time.

According to an embodiment, the hybrid molecules according to the invention can also be obtained by bioproduction methods and encoded by a recombinant DNA encoding the peptide according to the invention and the Fc fragment, said peptide and said Fc fragment being optionally separated by a polypeptide linker. In such a case, the peptide of the hybrid molecule is unmodified (e.g., non-cyclized, non-retro, non-retro-inverso), and may be N-terminal or C-terminal oriented. The peptide may be at the C-terminal or N-terminal end of the Fc fragment, with the cDNA including its nucleotide coding sequence upstream or downstream of the cDNA of the Fc.

The sequences according to the invention are shown in Table 1 below.

TABLE 1
Summary table of the sequences according to the invention

| Sequence number/ | |
|---|---|
| Sequence name if | |
| applicable | Sequence |
| SEQ ID NO: 1 | VVLGAHNV |
| SEQ ID NO: 2 | VLGAHNVR |
| SEQ ID NO: 3 | LGAHNVRT |
| SEQ ID NO: 4 | GAHNVRTQ |
| SEQ ID NO: 5 | PHSRPYMA |
| SEQ ID NO: 6 | QPHSRPYM |
| SEQ ID NO: 7 | HSRPYMAS |
| SEQ ID NO: 8 | FLNNYDAE |
| SEQ ID NO: 9 | CFGDSGGP |
| SEQ ID NO: 10 | MAHRPPSP |
| SEQ ID NO: 11 | AHRPPSPA |
| SEQ ID NO: 12 | HRPPSPAL |
| SEQ ID NO: 13 | AQPHSRPY |
| SEQ ID NO: 14 | PGSHFCGG |
| SEQ ID NO: 15 | PVPHGTQC |
| SEQ ID NO: 16 | FCRPHNI |
| SEQ ID NO: 17 | PRRKAGI |
| SEQ ID NO: 18 | ATVQLPQ |
| SEQ ID NO: 19 | QLPQQDQ |
| SEQ ID NO: 20 | PVPHGTQ |
| SEQ ID NO: 21 | QPHSRPY |
| SEQ ID NO: 22 | RVGAHDP |
| SEQ ID NO: 23 | IDSFVIW |
| SEQ ID NO: 24/ | APELLGGPSVFLFPPKPKDTLMISRTPEVTCVVVDVSHEDPEVKFNWYVDGVEVHNAKT |
| Fc Fragment | KPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 25/ | LLQGARSDATHTCPPCPAPELLGGPSVFLFPPKPKDTLMISRTPEVTCVVVDVSHEDP |
| Fc Fragment + | EVKFNWYVDGVEVHNAKTKPREEQYNSTYRVVSVLTVLHQDWLNGKEYKCKVSNKALP |
| Qtag | APIEKTISKAKGQPREPQVYTLPPSREEMTKNQVSLTCLVKGFYPSDIAVEWESNGQP |
| | ENNYKTTPPVLDSDGSFFLYSKLTVDKSRWQQGNVFSCSVMHEALHNHYTQKSLSLSP |
| | GK |
| SEQ ID NO: 26/ | AHGHGHGLLQGARSDATHTCPPCPAPELLGGPSVFLFPPKPKDTLMISRTPEVTCVVV |
| Fc Fragment + | DVSHEDPEVKFNWYVDGVEVHNAKTKPREEQYNSTYRVVSVLTVLHQDWLNGKEYKCK |
| Qtag bis | VSNKALPAPIEKTISKAKGQPREPQVYTLPPSREEMTKNQVSLTCLVKGFYPSDIAVE |
| | KWESNGQPENNYKTTPPVLDSDGSFFLYSKLTVDKSRWQQGNVFSCSVMHEALHNHYT |
| | QSLSLSPGK |
| SEQ ID NO: 27/ | LLQG |
| (Qtag example) | |
| SEQ ID NO: 28/ | MAHRPPSPALASVLLALLLSGAARAAEIVGGHEAQPHSRPYMASLQMRGNPGSHFCGG |
| Sequence of the | TLIHPSFVLTAAHCLRDIPQRLVNVVLGAHNVRTQEPTQQHFSVAQVFLNNYDAENKL |
| PR3 | NDVLLIQLSSPANLSASVATVQLPQQDQPVPHGTQCLAMGWGRVGAHDPPAQVLQELN |
| | VTVVTFFCRPHNICTFVPRRKAGICFGDSGGPLICDGIIQGIDSFVIWGCATRLFPDF |
| | FTRVALYVDWIRSTLRRVEAKGRP |
| SEQ ID NO: 29/ | MGVPFFSSLRCMVDLGPCWAGGLTAEMKLLLALAGLLAILATPQPSEGAAPAVLGEVDT |
| Sequence of the | SLVLSSMEEAKQLVDKAYKERRESIKQRLRSGSASPMELLSYFKQPVAATRTAVRAADY |
| MPO | LHVALDLLERKLRSLWRRPFNVTDVLTPAQLNVLSKSSGCAYQDVGVTCPEQDKYRTIT |
| | GMCNNRRSPTLGASNRAFVRWLPAEYEDGFSLPYGWTPGVKRNGFPVALARAVSNEIV |
| | RFPTDQLTPDQERSLMFMQWGQLLDHDLDFTPEPAARASFVTGVNCETSCVQQPPCFPL |
| | KIPPNDPRIKNQADCIPFFRSCPACPGSNITIRNQINALTSFVDASMVYGSEEPLARN |
| | LRNMSNQLGLLAVNQRFQDNGRALLPFDNLHDDPCLLTNRSARIPCFLAGDTRSSEMPE |
| | LTSMHTLLLREHNRLATELKSLNPRWDGERLYQEARKIVGAMVQIITYRDYLPLVLGPT |
| | AMRKYLPTYRSYNDSVDPRIANVFTNAFRYGHTLIQPFMFRLDNRYQPMEPNPRVPLSR |
| | VFFASWRVVLEGGIDPILRGLMATPAKLNRQNQIAVDEIRERLFEQVMRIGLDLPALNM |
| | QRSRDHGLPGYNAWRRFCGLPQPETVGQLGTVLRNLKLARKLMEQYGTPNNIDIWMGGV |
| | SEPLKRKGRVGPLLACIIGTQFRKLRDGDRFWWENEGVFSMQQRQALAQISLPRIICDN |
| | TGITTVSKNNIFMSNSYPRDFVNCSTLPALNLASWREAS |
| SEQ ID NO: 30/ | MKLVRFLMKLSHETVTIELKNGTQVHGTITGVDVSMNTHLKAVKMTLKNREPVQLETLS |
| Sequence of the | IRGNNIRYFILPDSLPLDTLLVDVEPKVKSKKREAVAGRGRGRGRGRGRGRGRGRGGP |
| SmD1 | RR |
| SEQ ID NO: 31 | AHCLRDIPQRLVNV |
| SEQ ID NO: 32 | HGTQCLAMGWGRVGAH |
| SEQ ID NO: 33 | NICTFVPRRKAGIC |
| SEQ ID NO: 34 | SRPYMA |
| SEQ ID NO: 35 | ATRLFPDFFTRVALY |
| SEQ ID NO: 36 | VEPKVKSKKREAVAGRGRGRGRGRGRGRGRGRGGPRR |
| SEQ ID NO: 37 | VAGRGRGRGRGRGRGRGRGRGGPRR |
| SEQ ID NO: 38 | MPELTSMHTLLLREH |
| SEQ ID NO: 39 | PILRGLMATPAKLNR |
| SEQ ID NO: 40 | DHGLPGYNAWR |
| SEQ ID NO: 41 | FPTDQLTPDQER |
| SEQ ID NO: 42 | IANVFTNAFR |
| SEQ ID NO: 43 | IGLDLPALNMQR |
| SEQ ID NO: 44 | IPCFLAGDTR |
| SEQ ID NO: 45 | LFEQVMR |
| SEQ ID NO: 46 | NNIFMSNTYPR |
| SEQ ID NO: 47 | RKIVGAMVQIITY |
| SEQ ID NO: 48 | RSPTLGASNR |
| SEQ ID NO: 49 | VFFASWR |
| SEQ ID NO: 50 | VVLEGGIDPILR |
| SEQ ID NO: 51 | RKIVGAMVQIITYRD |
| SEQ ID NO: 52 | GHEAQPHSRPYMAS |
| SEQ ID NO: 53 | NVVLGAHNVRTQE |
| SEQ ID NO: 54 | ATVQLPQQDQPVPHGTQ |
| SEQ ID NO: 55 | HGTQCLAMGWGRVGAHD |

Other features, details and advantages of the invention will appear on reading the attached Figures and examples which illustrate the invention and are not intended to limit it in any way.

### BRIEF DESCRIPTION OF THE FIGURES

FIG. 1 shows the outline of an example of a hybrid molecule according to the invention.

A spacer (which is optional) is shown between the antibody Fc fragment and the peptide bonding to an autoreactive lymphocyte involved in autoimmune vasculitis (called “peptide according to the invention”).

FIG. 2 represents a B-lymphocyte expressing on its surface transmembrane autoantibodies/BCR bonded to a hybrid molecule according to the invention, said hybrid molecule itself being bonded by the Fc fragment to an NK-cell.

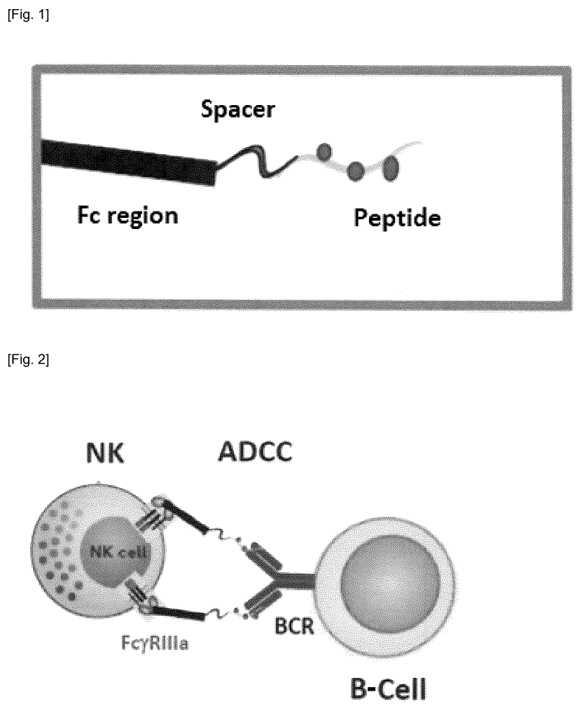

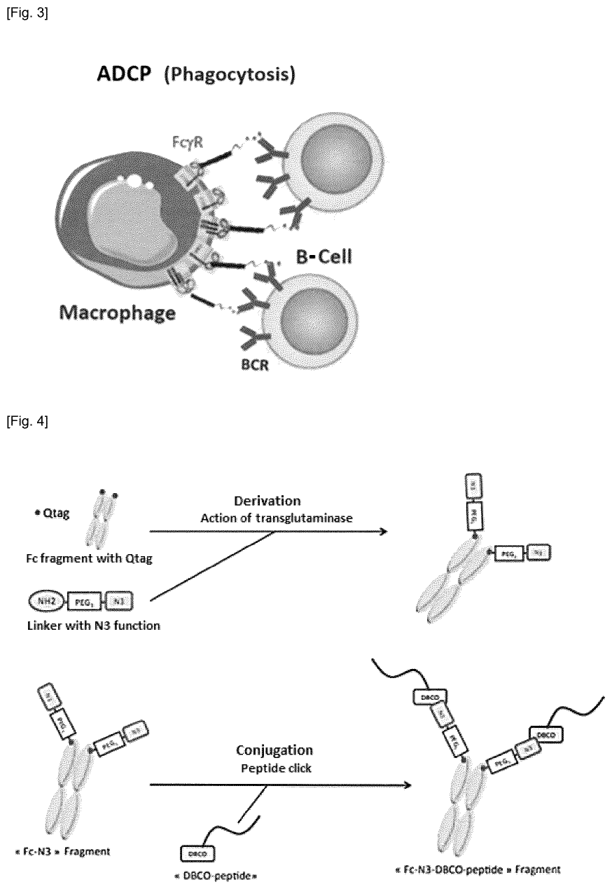

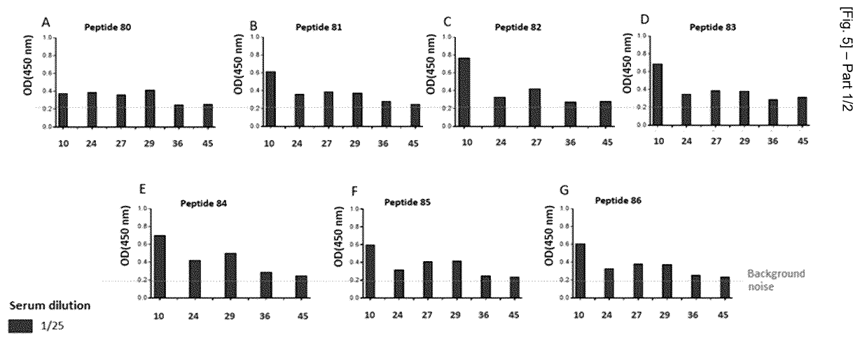

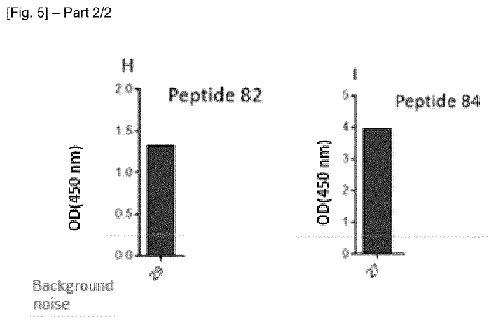

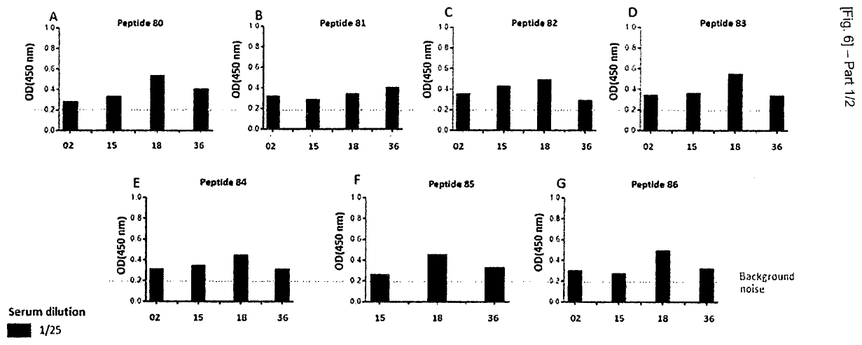

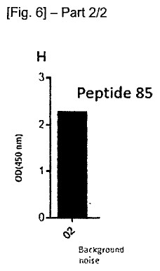

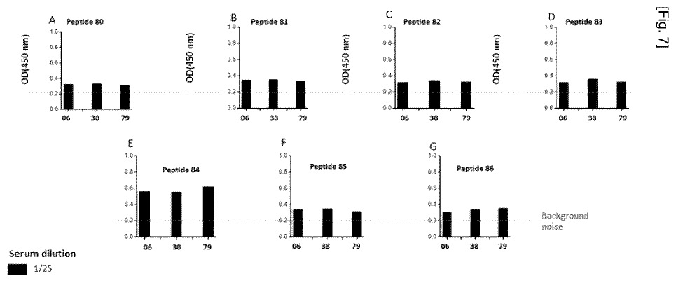

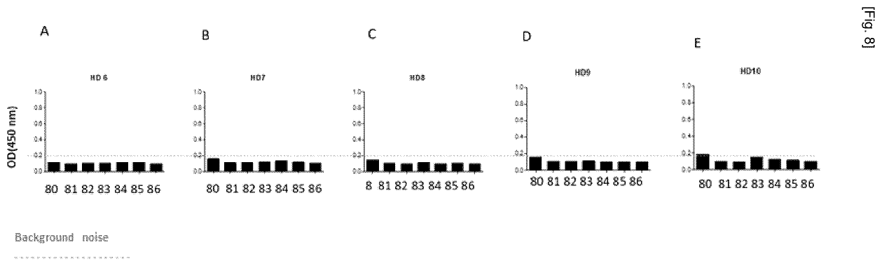

The NK-cell will thus be able to destroy the B-lymphocyte by ADCC. More specifically, this Figure shows a B-cell (or B-lymphocyte) which expresses on its surface a BCR (the ANCA autoantibody) which specifically interacts with the peptide according to the invention carried by the hybrid molecule. The connection of the Fc fragment carried by the same hybrid molecule to FcγRIIIa (CD16a) expressed on the surface of an NK-cell will activate ADCC and induce the specific destruction of the “ANCA positive” B-lymphocyte (which expresses an ANCA). (ADCC, Antibody-Dependent Cell Cytotoxicity; BCR, B-Cell Receptor; FcγR, Fc-gamma Receptor; NK cell, Natural Killer cell).

NB: a similar representation could be achieved by replacing the B-lymphocyte with a T-lymphocyte, and the BCR with a TCR (T-Cell Receptor).

FIG. 3 represents a B-lymphocyte expressing on its surface transmembrane BCR bonded to a hybrid molecule according to the invention, said hybrid molecule itself being bonded by the Fc fragment to a macrophage.

The macrophage will thus be able to destroy the B-lymphocyte by phagocytosis. More specifically, this Figure shows B-cells (or B-lymphocytes) which express on their surface a BCR (the ANCA autoantibody) and which specifically interact with peptides according to the invention carried by the hybrid molecules. The engagement of Fc fragments carried by these same hybrid molecules to different FcγRs expressed on the surface of a macrophage will activate ADCP, i.e. phagocytosis, and induce the specific destruction of “ANCA positive” B-lymphocytes. (ADCP, Antibody-Dependent Cellular Phagocytosis; BCR, B-Cell Receptor; FcγR, Fc-gamma Receptor).

NB: a similar representation could be achieved by replacing the B-lymphocyte with a T-lymphocyte, and the BCR with a TCR (T-Cell Receptor).

FIG. 4 represents a method for manufacturing a hybrid molecule according to the invention, comprising the derivation and conjugation outline of the Fc fragments.

FIG. 5 represents the reactivity of sera from patients with micropolyangiitis in the presence of peptides. The reactivity of the sera in the presence of peptide 80 is shown in FIG. 5A, the reactivity of the sera in the presence of peptide 81 is shown in FIG. 5B, the reactivity of the sera in the presence of peptide 82 is shown in FIGS. 5C and 5H, the reactivity of the sera in the presence of peptide 83 is shown in FIG. 5D, the reactivity of the sera in the presence of peptide 84 is shown in FIGS. 5E and 5I, the reactivity of the sera in the presence of peptide 85 is shown in FIG. 5F, and the reactivity of the sera in the presence of peptide 86 is shown in FIG. 5G. In the graphs, the ordinate represents the OD measured at 450 nm and the abscissa represents the tested serum. The serum is diluted 1/25.

FIG. 6 represents the reactivity of sera from patients with Wegener's disease in the presence of peptides. The reactivity of the sera in the presence of peptide 80 is shown in FIG. 6A, the reactivity of the sera in the presence of peptide 81 is shown in FIG. 6B, the reactivity of the sera in the presence of peptide 82 is shown in FIG. 6C, the reactivity of the sera in the presence of peptide 83 is shown in FIG. 6D, the reactivity of the sera in the presence of peptide 84 is shown in FIG. 6E, the reactivity of the sera in the presence of peptide 85 is shown in FIGS. 6F and 6H, and the reactivity of the sera in the presence of peptide 86 is shown in FIG. 6G. In the graphs, the ordinate represents the OD measured at 450 nm and the abscissa represents the tested serum. The serum is diluted 1/25.

FIG. 7 represents the reactivity of sera from patients with Churg-Strauss syndrome in the presence of peptides. The reactivity of the sera in the presence of peptide 80 is shown in FIG. 7A, the reactivity of the sera in the presence of peptide 81 is shown in FIG. 7B, the reactivity of the sera in the presence of peptide 82 is shown in FIG. 7C, the reactivity of the sera in the presence of peptide 83 is shown in FIG. 7D, the reactivity of the sera in the presence of peptide 84 is shown in FIG. 7E, the reactivity of the sera in the presence of peptide 85 is shown in FIG. 7F, and the reactivity of the sera in the presence of peptide 86 is shown in FIG. 7G. In the graphs, the ordinate represents the OD measured at 450 nm and the abscissa represents the tested serum. The serum is diluted 1/25.

FIG. 8 represents the reactivity of sera from healthy patients in the presence of peptides. The reactivity of serum HD6 in the presence of peptides 80-86 is shown in FIG. 8A, the reactivity of serum HD7 in the presence of peptides 80-86 is shown in FIG. 8B, the reactivity of serum HD8 in the presence of peptides 80-86 is shown in FIG. 8C, the reactivity of serum HD9 in the presence of peptides 80-86 is shown in FIG. 8D, and the reactivity of serum HD10 in the presence of peptides 80-86 is shown in FIG. 8E. In the graphs, the ordinate represents the OD measured at 450 nm and the abscissa represents the tested peptide. The serum is diluted 1/25.

### EXAMPLES

### Example 1: Example of Production of a Hybrid Molecule According to the Invention

The hybrid molecule described here comprises the following structure: an Fc fragment covalently bonded to at least one PEGn spacer, itself coupled to an azide, said azide being covalently bonded to an alkyne, which is itself coupled to a PEGn spacer bonded to a peptide according to the invention. Such a molecule can be read: Fc-PEGn-N₃-DBCO-PEGn-peptide. The two PEGn may be the same or different (for example the first PEGn is PEGs and the second PEGn is PEG₂).

1. Synthesis of a NH₂-PEGn-N₃ (i.e., a spacer coupled to an azide at one end and having a free amine function at another end).

2. Placement of NH₂-PEGn-N₃, with an Fc fragment having a Qtag, and a transglutaminase, preferably for 16 hours at 37 °C. This so-called “derivation” step is, for example, carried out with 20 times more moles of NH₂-PEGn-N₃ than moles of Fc fragment. Transglutaminase is used, for example at a level of 15 U/μmol per present Qtag (on one or both monomers). Optionally, desalting may be carried out to remove excess spacer not bonded to the Fc fragment at the end of the step. A Fc-PEGn-N₃ is thus obtained. If the Fc fragment has 2Qtags (one carried on each monomer), then the Fc fragment can carry two PEGn-N₃.

3. Synthesis of a Cys-PEGn-peptide (i.e., a spacer bonded to a peptide at one end and having a free cysteine at another end). An alkyne, for example DBCO, is then coupled to the Cys-PEGn-peptide at the cysteine, and a DBCO-PEGn-peptide is thus obtained.

4. Achieving the “click”: coupling between the N₃ and the DBCO. The DBCO-PEGn-peptide is placed with the Fc-PEGn-N₃ with, preferably, 10 times more moles DBCO-PEGn-peptide than moles Fc-PEGn-N₃. The click reaction takes place in particular at room temperature and is almost complete after 4 hours.

5. Obtaining the hybrid molecule: Fc-PEGn-N₃-DBCO-PEGn-peptide. This embodiment is illustrated in FIG. 4.

Similarly, a Fc-PEGn-DBCO and a N₃-PEGn-peptide can be obtained, then coupled to obtain Fc-PEGn-DBCO-N₃-PEGn-peptide.

Similarly, a Fc-PEGn-DBCO and an N₃-peptide can be obtained, or a Fc-PEGn-N₃ and a DBCO-peptide, then coupled to obtain Fc-PEGn-DBCO-N₃-peptide or Fc-PEGn-N₃-DBCO-peptide, respectively.

### Example 2: Reactivity of Sera from Patients with Autoimmune Vasculitis to Hybrid Molecules According to the Invention

The sera used are sera from patients with autoimmune vasculitis, more particularly granulomatosis with polyangiitis, which contain specific autoantibodies.

ELISA microtiter plate wells were coated by passive adsorption using 100 μL of a solution containing either a peptide selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50 and SEQ ID NO: 51, or a hybrid molecule according to the invention, wherein the Fc fragment is wild-type (WT) or the Fc fragment comprises the GASDIE mutation. Such a hybrid molecule is for example “FcWT-PEG-SEQ ID NO: 1-23/31-51”, “FcLALAPG-PEG-SEQ ID NO: 1-23/31-51” or “FcGASDIE-PEG-SEQ ID NO: 1-23/31-51». «FCWT-PEG-SEQ ID NO: 1-23/31-51” represents a wild-type Fc fragment which is bonded to at least one PEGn spacer which is itself coupled to an azide covalently bonded to a DBCO cyclooctyne coupled to a PEGn spacer which is itself bonded to a peptide represented by any one of SEQ ID NO: 1 to SEQ ID NO: 23 or SEQ ID NO: 31 to SEQ ID NO: 51, “FcWT-LALAPG-SEQ ID NO: 1-23/31-51” represents an Fc fragment, comprising the L234A, L235A and P329G mutations, which is bonded to at least one PEGn spacer which is itself coupled to an azide covalently bonded to a DBCO cyclooctyne coupled to a PEGn spacer which is itself bonded to a peptide represented by any one of SEQ ID NO: 1 to SEQ ID NO: 23 or SEQ ID NO: 31 to SEQ ID NO: 51, and “FcGASDIE-PEG-SEQ ID NO: 1-23/31-51” represents an Fc fragment, comprising the G236A, S239D and 1332E mutations, which is bonded to at least one PEGn spacer which is itself coupled to an azide covalently bonded to a DBCO cyclooctyne coupled to a PEGn spacer which is itself bonded to a peptide represented by any one of SEQ ID NO: 1 to SEQ ID NO: 23 or SEQ ID NO: 31 to SEQ ID NO: 51. In the structures, n represents a number between 1 and 10 when a PEGn spacer is used, preferably 1, 2, 3, 4 or 8.

These solutions were each used at a concentration of 5 μg/mL in a PBS (Phosphate Buffered Saline) buffer and incubated overnight at 4 °C. The selected peptides and the hybrid molecules constructed with said peptides synthesized in scramble sequence (in French: “séquence aléatoire”) were used at the same concentration as negative controls. After washing in PBS Tween 0.05%, the wells were then saturated with 5% milk for 1 hour at room temperature. After washing, the sera were incubated in serial dilution in PBS buffer+5% milk for 1 hour at room temperature. After washing, the reactivity of the sera was detected using an anti-Fab detection antibody for detecting human IgG coupled with HRP diluted 1/2500 in PBS buffer 5% milk also incubated for 1 hour at room temperature. After washing, the presence of autoantibodies recognized by the hybrid molecule is revealed by adding TMB and stopping the enzymatic reaction by adding H2SO4 and reading on the spectrophotometer at 450 nm.

The results are expressed in Δ DO (Delta-Optical Density), corresponding to the DO obtained with a scramble hybrid peptide or with the hybrid molecule constructed with said peptide.

The results show a dose-dependent reactivity of sera on peptides with respect to hybrid molecules. They show that the peptides, when they are included in the different hybrids, remain perfectly reactive to the sera including the autoantibodies.

### Example 3: Binding Kinetics of the Hybrid Molecules onto Macrophages, at Physiological Temperature

Human macrophages, differentiated in vitro in the presence of M-CSF (100 ng/ml) from CD14+ monocytes isolated from peripheral blood of a healthy subject were incubated at 500,000 cells/well, in the presence of hybrid molecules at 5 μg/mL. Hybrid molecules are for example those described in Example 2.

The hybrid molecules are incubated for 2 h, 8 h, 16 h, 24 h and 48 h at 37 °C. Binding of the hybrid molecules to the surface of macrophages is demonstrated and quantified by cytofluorimetry (FACS Canto II) after incubating the macrophages with an anti-Fc antibody coupled to FITC, used 1/1000.

“NM” and “ANTI FC” represent, respectively, the well containing macrophages incubated without antibody or with the sole anti-Fc antibody. “Wt”, “LALAPG” and “GASDIE” represent respectively the well containing the hybrid molecule with the wild-type Fc fragment or with the LALAPG mutation or with the GASDIE mutation.

The results show that the hybrid molecules at the physiological temperature of 37 °C, bind to the membrane of macrophages. These results also show that the binding of the hybrid comprising an Fc fragment comprising the GASDIE mutation is greater than that of the wild form.

### Example 4: Phagocytosis Induced by the Fc-WT Hybrid or the Fc-GASDIE Hybrid when Arming B-Lymphocytes Via their FcγRIIb (CD32b) in the Presence of Sera

Freshly purified human B-lymphocytes from peripheral blood of healthy donors by CD19+magnetic sorting were labeled with green PKH-67 (Paul Karl Horan/fluorescent lipid marker), then incubated with sera from patients with autoimmune vasculitis at a concentration of 5 μg/mL for 30 min at 37 °C. Subsequently, the cells were washed, then placed with the hybrid according to the invention comprising a wild-type Fc fragment (Wt) or comprising the GASDIE mutations. Hybrid molecules are for example those described in Example 2. The cells were placed in the presence of the hybrids at 10 μg/mL for 30 min at 37 °C. After washing, the cells were placed in contact with the macrophages (marked with red PKH-26), at a rate of 1 B-lymphocyte for 1 macrophage, for 2 hours at 37 °C. The cells were then detached and analyzed by flow cytometry. Cells showing double fluorescence (green PKH-67/red PKH-26) correspond to macrophages that have phagocytosed lymphocytes. The different cell populations are expressed as a percentage of the total cell population: percentage of phagocytosis and percentage of B-lymphocytes.

The obtained results confirm the phagocytosis activity is increased in the presence of hybrids (increase in phagocytosis associated with a decrease in the B-lymphocyte population). Indeed, a significant increase in the percentage of phagocytosis, always associated with a significant decrease in the B-lymphocyte population, was observed when the B-lymphocytes were coated with the hybrid.

### Example 5: Binding Stability of Fc-GASDIE A488 and of the Fc-GASDIE Peptide Hybrids on NK-Cells after 30 Min, 24 h and 48 h, at Physiological Temperature

The NK cells were incubated for 30 min, 24 h or 48 h at 37 °C in the presence of FcGASDIE-N3-DBCO-A488 (an Fc fragment comprising the G236A, S239D and 1332E mutations which is bonded to at least one PEGn spacer which is itself coupled to an azide covalently bonded to a DBCO, the molecule being labeled with fluorochrome A488) or of FcGASDIE-N3-DBCO-PEG3-peptide-lysineA488 (an Fc fragment comprising the G236A, S239D and 1332E mutations which is bonded to at least one PEGn spacer which is itself coupled to an azide covalently bonded to a DBCO which is coupled to a PEGn spacer bonded to a peptide according to the invention (represented by any one of SEQ ID NOs: 1 to SEQ ID NO: 23 or SEQ ID NO: 31 to SEQ ID NO: 51) the molecule being labeled with fluorochrome A488 which is bonded to the molecule via a lysine). Binding of these 2 fluorescent probes to NK cells was analyzed by cytofluorimetry. In the structures, n represents a number between 1 and 10 when a PEGn spacer is used, preferably 1, 2, 3, 4 or 8.

Results are shown at 30 minutes, 24 hours and 48 hours following. NM represents unlabeled NK-cells, without antibodies.

### Example 6: Reactivity of Sera with Peptides According to the Invention

The peptides used herein are the peptides of SEQ ID NO: 52, SEQ ID NO: 53, SEQ ID NO: 54, SEQ ID NO: 2, SEQ ID NO: 31, SEQ ID NO: 55 and SEQ ID NO: 7, as indicated in Table 1 above.

The peptide represented by SEQ ID NO: 52 corresponds to the peptide named peptide 80 in the examples. The peptide represented by SEQ ID NO: 53 corresponds to the peptide named peptide 81 in the examples. The peptide represented by SEQ ID NO: 54 corresponds to the peptide named peptide 82 in the examples. The peptide represented by SEQ ID NO: 2 corresponds to the peptide named peptide 83 in the examples. The peptide represented by SEQ ID NO: 31 corresponds to the peptide named peptide 84 in the examples. The peptide represented by SEQ ID NO: 55 corresponds to the peptide named peptide 85 in the examples. The peptide represented by SEQ ID NO: 7 corresponds to the peptide named peptide 86 in the examples.

The peptides were synthesized and their reactivity was tested with serum from different patients.

The sera used here are shown in Table 2 below.

TABLE 2
Tested sera

| Name of serum | Characteristics of the serum |
|---|---|
| 45 | Serum from a patient with micropolyangiitis, obtained from Central BioHub |
| 10 | Serum from a patient with micropolyangiitis, obtained from Central BioHub |
| 27 | Serum from a patient with micropolyangiitis, obtained from Central BioHub |
| 24 | Serum from a patient with micropolyangiitis, obtained from Central BioHub |
| 29 | Serum from a patient with micropolyangiitis, obtained from Central BioHub |
| 36 | Serum from a patient with micropolyangiitis, obtained from Central BioHub |
| 6 | Serum from a patient with Churg-Strauss syndrome, obtained from Central BioHub |
| 38 | Serum from a patient with Churg-Strauss syndrome, obtained from Central BioHub |
| 79 | Serum from a patient with Churg-Strauss syndrome, obtained from Central BioHub |
| 36 | Serum from a patient with Wegener's disease, obtained from Central BioHub |
| 2 | Serum from a patient with Wegener's disease, obtained from Central BioHub |
| 18 | Serum from a patient with Wegener's disease, obtained from Central BioHub |
| 15 | Serum from a patient with Wegener's disease, obtained from Central BioHub |
| HD6 | Serum from a healthy patient, from a blood donation. The serum was collected |
| | after a 30-minute centrifugation on Ficoll, aliquoted and frozen at −20 °C. |
| HD7 | Serum from a healthy patient, from a blood donation. The serum was collected |
| | after a 30-minute centrifugation on Ficoll, aliquoted and frozen at −20 °C. |
| HD8 | Serum from a healthy patient, from a blood donation. The serum was collected |
| | after a 30-minute centrifugation on Ficoll, aliquoted and frozen at −20 °C. |
| HD9 | Serum from a healthy patient, from a blood donation. The serum was collected |
| | after a 30-minute centrifugation on Ficoll, aliquoted and frozen at −20 °C. |
| HD10 | Serum from a healthy patient, from a blood donation. The serum was collected |
| | after a 30-minute centrifugation on Ficoll, aliquoted and frozen at −20 °C. |

ELISA microtiter plate wells were coated by passive adsorption using 100 μL of a solution containing a peptide represented by SEQ ID NO: 52, SEQ ID NO: 53, SEQ ID NO: 54, SEQ ID NO: 2, SEQ ID NO: 31, SEQ ID NO: 55 or SEQ ID NO: 7.

These solutions were each used at a concentration of 10 μg/mL in a PBS (Phosphate Buffered Saline) buffer and incubated overnight at 4 °C. The next day, the wells were saturated with a blocking buffer with 200 μg/mL per well for 1 h at room temperature. After washing, the sera were incubated, diluted 1/25 with a buffer including PBS. They were incubated at 100 μg/mL per well for 2.5 hours at 4 °C, with orbital shaking. After washing, the reactivity of the sera was detected using a human IgG anti-Fc detection antibody coupled to HRP, diluted 1/5000 in PBS-2% BSA buffer, and also incubated for 1 hour at 4 °C with orbital shaking. After washing, the disclosure is carried out by adding TMB and stopping the enzymatic reaction by adding sulfuric acid and a reading on the spectrophotometer at 450 nm.

The results express the optical density (OD) obtained with each serum and each tested peptide.

The background noise was obtained by following the same protocol described above but without the first peptide coating step. This thus represents the non-specific adhesion of IgG present in the serum of patients, this value being equivalent whatever the pathology.

The reactivity of peptides 80, 81, 82, 83, 84, 85 and 86 with sera from patients with micropolyangiitis (sera 10, 24, 27, 29, 36 and 45) is shown in FIG. 5.

The reactivity of peptides 80, 81, 82, 83, 84, 85 and 86 with sera from patients with Wegener's disease (sera 2, 15, 18 and 36) is shown in FIG. 6.

The reactivity of peptides 80, 81, 82, 83, 84, 85 and 86 with sera from patients with Churg-Strauss syndrome (sera 6, 38 and 79) is shown in FIG. 7.

The reactivity of peptides 80, 81, 82, 83, 84, 85 and 86 with sera from healthy patients (sera HD6, HD7, HD8, HD9 and HD10) is shown in FIG. 8.

The results thus confirm that sera from healthy donors are not reactive in the presence of the tested peptides. In contrast, sera from patients with micropolyangiitis, Wegener's disease, or Churg-Strauss syndrome react in the presence of the tested peptides. These results thus illustrate that the peptides according to the invention are recognized by the autoantibodies present in the sera of patients suffering from micropolyangiitis, Wegener's disease or Churg-Strauss syndrome.

1: A hybrid molecule comprising at least one antibody Fc fragment covalently bonded to at least one peptide bonding to an autoreactive lymphocyte involved in autoimmune vasculitis, at least one spacer being optionally present between said Fc fragment and said peptide.

2: A hybrid molecule according to claim 1, in which said peptide comprises all or part of the polypeptide sequence of proteinase 3, all or part of the polypeptide sequence of myeloperoxidase and/or all or part of the polypeptide sequence of the small nuclear ribonucleoprotein SmD1.

3: A hybrid molecule according to claim 1, wherein the sequence is of human origin.

4: A hybrid molecule according to claim 1, in which said spacer is a polymer containing one or more repeating units containing the ether group.

5: A hybrid molecule according to claim 1, wherein said peptide is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 and SEQ ID NO: 55.

6: A hybrid molecule according to claim 1, in which said Fc fragment is a human Fc fragment.

7: A hybrid molecule according to claim 1, wherein said Fc fragment is a wild-type or mutated fragment, said mutated Fc fragment preferably comprising at least the following mutations:
L234A and L235A, or
L234A, L235A and P329G, or
G236A, S239D and 1332E, or
G236A, S239D, A330L and 1332E, or
S239D, H268F, S324T and 1332E,
the numbering being indicated in the sequence of a human IgG1 according to the EU index.

8: A hybrid molecule according to claim 1, in which:
the Fc fragment is coupled to at least one azide and said peptide is coupled to an alkyne, or
the Fc fragment is coupled to at least one alkyne, and said peptide is coupled to an azide, or
the Fc fragment is coupled to at least one azide and said peptide is bonded to a spacer itself coupled to an alkyne, or
the Fc fragment is coupled to at least one alkyne, and said peptide is bonded to a spacer itself coupled to an azide or
the Fc fragment is bonded to at least one spacer, itself coupled to an azide, and said peptide is coupled to an alkyne, or
the Fc fragment is bonded to at least one spacer, itself coupled to an alkyne, and said peptide is coupled to an azide or
the Fc fragment is bonded to at least one spacer, itself coupled to an azide, and said peptide is bonded to a spacer, itself coupled to an alkyne, or
the Fc fragment is bonded to at least one spacer, itself coupled to an alkyne, and said peptide is bonded to a spacer, itself coupled to an azide,
wherein the covalent bond between said Fc fragment and said peptide, optionally in the presence of one or more spacers, is created between the azide and the alkyne.

9: A method for treating autoimmune vasculitis comprising administering a therapeutically effective amount of a hybrid molecule according to claim 1.

10: The method according to claim 9 hybrid molecule for its use according to claim 1, for the treatment of granulomatosis with polyangiitis, eosinophilic granulomatosis with polyangiitis or micropolyangiitis.

11: A hybrid molecule according to claim 4, in which said spacer is polyethylene glycol of formula PEGn, wherein n denotes an integer between 1 and 100.

12: A hybrid molecule according to claim 11, in which said spacer is polyethylene glycol of formula PEGn, wherein n is 1, 2, 3, 4 or 8.

13: A hybrid molecule according to claim 1, in which said Fc fragment is a human Fc fragment of IgG, more particularly of IgG1.

14: A hybrid molecule according to claim 1, in which said Fc fragment is a human Fc fragment of IgG1.

15: A hybrid molecule according to claim 8, in which said alkyne is cyclooctyne.

16: A hybrid molecule according to claim 15, in which said cyclooctyne is DBCO.
